SEQUENCE LISTING (12 sequences)

SEQ ID NO: 1 - DNA, 849 bp, synthetic construct
  misc_feature 1..849: note = GT062
  source 1..849: mol_type = other DNA
atgatcatcgttcgtctgtcggatggcctgggtaaccagatgttccagtacgcgttcggtcgtgcgctgagcacccgccgtggtgttccgctgcgtctggacgtttccgcataccgcgtagaacgtaaacgtcgttacgaactgcaccactttctgaccgaagaaaccttcgttaccgatgaggaagcgcaccgtgttatcacccgtccgcattccccggacgaaccgtggtggtcccagccggttgttcgtgaaccgcacttccactatagcccggatgttgttcaggtttcgtccgcgggttacttcgcgggctactggcagtctgaacgtcacttcgatgatgttgcgccgctgatccgcctggaatttaccccgaaacagccgctgaccggcgcgaacctggaagttgcgcgtgcgatcgcggcgcgtaacgcggttagcctgcacgttcgtcgtggtgattacatctgcgatccgaaagttaacatcctgcacggcgtttgctctctggaatactaccgtgcggcggttgcgtacgttgcggcgcgtgttgaaaaaccggaatttttcgttttcaccgatgatccggattggacccgtaccaaactgaaactggatttcccggcgtacctggtgacccagaaccaggatgcgccggttgaagacctgcgcctgatgaccctgtgccgtcaccacatcatcgcgaactcttctttctcctggtggggtgcttggctgggtgaaaaaccgggtcagatcgtttgcgcgccgcagcgttggttcggtgcgtacccgcacgatacccgtgatctggttccggatcgttggacccgtctggatggttaa

SEQ ID NO: 2 - DNA, 858 bp, synthetic construct
  misc_feature 1..858: note = GT065
  source 1..858: mol_type = other DNA
atgcagatcatctggtgcagcggcggcctgggcaaccagatgttccagtacgcgttctaccgtcgtctgcaactggacggcaaaagcgtgaccctggacatcagcggtttcaacgactacggcctgcacaacggcttcgaactggataaaatcttcccggtgaaaatcaacctggcggacgaagtgctgatcaacaacatcaaacagaaaatctctcacctgagcctgctgaaaaagatctggtggaaagtgttcaccaacttccgtccggtgatcgtgcagaaaaacttcggctacagtagccgtctgtctaacttgcagggtctgaaatacctggaaggctactggcagagcgaaaaatacttcggcacccacagcgacaccatccgtaacgacttcaaattcccgctgctggacattaaaaacaaagactacgccgacaaaattagccagggcgaagcggtgagcatccacatccgtatgggcgactacgtgaaccacccgctgcacggtggcatctgcaccctggaatactacaaaaaagcgctgagcctgattgaagaaaaagtagaaagcccgctgttcttcatctttagcaacgacattgaatggtgccagaacaacctgaaactggacaaagcgatctacgtgaccggtaacgaaggcaaaaacagcttccgtgatatgcacctgatgtccatgtgcaaacacaacatcatcgcgaacagctccttctcttggtggggcgcgtggctgaacaacaacccggacaaagttgttgtggcgccgagcaaatggttcaacgacaaaaccatcaacaccaaagatctgctgccggacagctggatccagatctaa

SEQ ID NO: 3 - DNA, 882 bp, synthetic construct
  misc_feature 1..882: note = GT072
  source 1..882: mol_type = other DNA
atgatcatcgttaacctgatgggcggcctgggcaaccagatgttccagtacgcgctgggccgccgtctggcgcaggataagggcgttgcgctgaaactggacacccagtggttcggcaaacagaacctgcgcaaattcgagctggacaaattcaacatcaccttcaaaatcgcatccgaagaagaaatctacagcacccgtcacttcttccgtaaccgtatcatccgcaaagtctacagcatctaccagaaccagctgccgtacttcaaacgttctttcatcaacgaaccgaacttcggtttcttcgatcaccacatcctggaagtgccgaaaaactgctacctgaccggctactggcagtccgaaaaatacttcagcagcatggaagataccatccgcaaagagttcaccctgaaagaaatcgcggactccaacttcatcgagttgagcaaagaaatgcagaacatcaactccgtgagcctgcacgtgcgccgcggcgactacgttaccaacccgcagaccaacaaattccacggcgtgctgagcaccgattactacaaactggcggtgaaactgatccagaacaaaatcgacaccccgcacttctacgtgttcagcgacgacctggaatgggttaaggaacgcctgaacttcgtgaccccgtgcacctacatcgaaggcaaaaaagaaggccgtgattgcgaagaaatgtggctgatgagccagtgcaaacaccacatcatcgcgaacagcagcttctcctggtggggcgcgtggctgggcaacaaaccggacaaaatcgttatcagcccgaaccagtggttcgcggataaaaactataaagttccggacctgatcccggaaaaatggatccgtatctaa

SEQ ID NO: 4 - DNA, 909 bp, synthetic construct
  misc_feature 1..909: note = GT083
  source 1..909: mol_type = other DNA
atgcaggtggttgttaaaatcaacggcggcctgggtaaccagatgttccagtacgcggcgggtcgtgctacctctctgcgtttcaacagcgttctgcaaatcgaaaccatcttcttcaaagacatcctgaacgaaggtgaacacaaacgtcagtaccagctgaacatcttcccgaacatcgcggcgctggatttacaggaaatcagcccgaaaaaccgccacaaacagaaaaaatacatcaactcttctatctacaaagcggaaaactctctgcgtggcaaactgggtatcaaactggcttaccagcacatctgggaaaagaacctgctgacctacgacccgtctttccagcagtctaacaaaaaagcgcacctgacctacctgatcggtgactggcagaacgaacagtacttcgaaagcgttgcggcgatcatccgcaacgatttcagcttcccgaccatcgaaagcggcagcctgaacgcggacatcctgtctcagatctacgcgagcgaagcggtggcggttcacgtgcgtcgcggtgattacctgctgccgggtatccactccccggtgagcccggcttactaccaggaagctctgagcctgatccgttcgaaagttgcgagcccgaaattcttcgttttctctgatgacatcaactggtgccgtgctaacctgggtctggcggacgcgtgcttcgttgaacacaacaccggcaccaacaactatcgtgatatgcagctgatgtcctcttgcaaacacaacatcatcgcgaactccagcttctcttggtggggcgcgtggctgaacaacaacccgaccaaaatcgttatcgcgccgagcatgtggatgccgacccaggcggttgaatctagccgtgttgttccgctgagctggatcaccctgtaa

SEQ ID NO: 5 - DNA, 867 bp, synthetic construct
  misc_feature 1..867: note = GT093
  source 1..867: mol_type = other DNA
atgaaaatcgttaaaatcctgggcggcctgggtaaccagatgttccagtacgcgctgtacctgtccttacaggaaaccttcccggaagaacgtgtgatgctggatctgagctgcttccagggctaccacctgcacaacggtttcgaactggaaaaaatcttcagcatcaaaggcgaaaaagcgagcgcgagcgacatcatgcgtgttgcgtactactacccgaactacctgctgtggcgtatcggtaaacgtctgctgccgtgccgtaaaggcatgtgcctggaaagcagcaccctgcgtttcgatgaaaccgttctgaccaaagaaggcaaccgttacttcgatggttactggcaggatgaacgttacttcgcggcgtgccgtgaaaaagttctgaaagcgttcaccttcccggcgttcaaacgtgctgaaaacctgagcctgctggaaaaactggatgaaaactctgttagcctgcacgttcgtcgtggtgattacatcggtaacagcctgtaccagggtatctgcgatctggactactaccgtaccgcgatcgaaaaaatgtgctcttacgttaccccgagcctgttctgcgttttcagcaacgacatcgaatggtgccgtgaacacctggaacagtacatcaacgcgccggttgtttatgttacctggaacaccggcgcggaaagctaccgtgatatgcagctgatgtcttgctgcgcgcacaacatcatcgcgaactctagcttctcctggtggggcgcgtggctgaaccagaactctgataaagttgttatcgcgccgaaaaaatggctgaacatggaagaatgccatttcgcgctgccgagcagctggatcaaaatctaa

SEQ ID NO: 6 - DNA, 885 bp, synthetic construct
  misc_feature 1..885: note = GT104
  source 1..885: mol_type = other DNA
atgaaaagcggtaaatacaaagacaaactgatcatccgtttcaaaggtggcctgggcaaccagatgttccagtacgcgatgtactgcaaacagaaacatctgggcaaacaggtttgcgcggatgtgagcgcgtacactgaacgtgaaggctgtatgccgttcgttctgtgcgacgttttcccgcagatcagcctccagctggtgaaagatgaagaagcggcgtattacctggcggcgcagaacaagaaaaacatcctggataaagtgatcgcggcgttctggtggcaggaacgtgactacacctctgaaaaagaaaacggcgtgttcgataaacgtgtgttcagcctgaaaaaaggcttcctggatggctattggcagaccgaaaaatacttctccgacatccgtgaagaactgctgaaagatttccagttcgaagttgccgatagctcgctgaaaaaatacgcggataaaatccgtgacaacagcgttagcgttcacgttcgtcgtggtgattacctgaacttcccggacatctacggtggtatctgcggcatggactactacaaaaaagcgatggacttcttctgcgagaaaaacccggaaaccgttttctacgttttctctgatgacaaagaatgggttcagaaagcgttccgtgaatacaacgctgtggtggttgaaaaagacttcttcagcgactacgaagattggtacgacatgtacctgatgagccagtgcaaccacaacatcatcgcgaactccagcttctcctggtggggtgcgtggctgaaccagaacaaaaacaaaaaagttatctctccgggcaaatggttcaacggcgaaaaaaccagcgacatctggtgcccggaatggatccgtatgtaa

SEQ ID NO: 7 - DNA, 858 bp, synthetic construct
  misc_feature 1..858: note = GT107
  source 1..858: mol_type = other DNA
atggttatcgttcagctgtctggtggcctgggtaaccagatgttcgaatacgcgctgtacctgcgtctgaaatctatgggtaaagaagttctggttgatgataccacctgctacggtccgggccagcgtaccaaacagctggatgttttcggcgtttcttacggtgcggcggatgaacgtcagctgcgtcgtatgaccgatagcgcgatggacccgctgtctcgtgcgcgtcgtaaactgtctggtcgtcgtgatctgtcttaccgtgaagcgggttgcgatttcgatccgctggttctggaaaaagatccggcgctgttgcagggttgcttccagtctgaacgttacttcggcgaaatccgtgatcaggttcgtgaagcgtaccgtttccgtaacctggtgaccaaccgtcgtgttgaagaataccgtctgcgtatcctggagaaaaaaggtgcgtccgttgcggttcacctgcgtcgtggtgattacctggacccgaaatacgcgggtctgtaccagggtatctgcaccgatgcgtggtacggtgaagcgatccgtctgatgaaacagaaagttccgggtgcggcgttcttcttcttctctaacgatccggattgggttaaagaacgttacggtggtgcgggcaacgttaccgttgaaggtggttctgaagatgcgggttacgaagatctgtacctgatgagcctgtgcggtcaccagatcatcgcgaacagcagcttctcttggtggggcgcttggctgaacgaaaacccggataaaaccgttatcgcgccgaaacgttggctgaacggccgtagctgccgtgacatctacaccaaagaaatgaccctgctgtaa

SEQ ID NO: 8 - DNA, 913 bp, synthetic construct
  misc_feature 1..913: note = GT059
  source 1..913: mol_type = other DNA
atgatcatcgttaaaatgatgggcggtctgggtaaccagatgttccagtgggcgctgggccgtgcgctggcgatcaaaaacagcagcgaatttaaaatcgacgtgtacttcctgatcgagcgccagccgcgtaaaaacttcaccatccgtacctacgatctggacgttttcaaactgaacgcggagttcgcgaccaaaaaagaaatcgcgtactacccgatcccgaaattcggcaaatacggcattttcctggtgcacctgaaacagatgtggcgccgtagcatcaacaccaacggctacaactacctgatccagacccgctttgattacgacgaacagatcgacaacgctccggtcaacagctatctggaaggctatttccagaccgagcgctacttcgaaccgtactccgacatcatccgcaaagacttcgagttccgcgacgaactgagcgggaaagcgctggaaatcgcccagctgatcaacaaaacccagtctgtggcggtgcacatccgtcgcggcgattacgttaccaaccgccgcgccaacaaaacgcacggcgtactgggcaaagaatactacgacaaagcgatggaaaccatcgcgagcaaagttgaaagcccgcactacttcatcttcagcgatgataacgaatggtgccgtgaaaacttcgcgttcggcgaaaacatgaccatcatcgaagatgacatcaaaggtaacaaattccagttctctctgaacctgatgtctcagtgcaaacacgcgatcatcgcgaacagcagcttctcttggtggggcgcgtggctgagcgcgaacccgaacaaaatcgttatcggcccgcagaactggttcaaaaacaccgacctgaacgttaaagacatcatcccggaaaaatggctgcgcatctaagctt

SEQ ID NO: 9 - DNA, 903 bp, synthetic construct
  misc_feature 1..903: note = HpFucT
  source 1..903: mol_type = other DNA
atggcttttaaagtggtgcaaatttgtggggggcttgggaatcaaatgtttcaatacgctttcgctaaaagtttgcaaaaacaccttaatacgcccgtgctattagacactacttcttttgattggagcaataggaaaatgcaattagagcttttccctattgatttgccctatgcgaatgcaaaagaaatcgctatagctaaaatgcaacatctccccaagttagtaagagatgcactcaaatacataggatttgatagggtgagtcaagaaatcgtttttgaatacgagcctaaattgttaaagccaagccgtttgacttatttttttggctatttccaagatccacgatattttgatgctatatcctctttaatcaagcaaaccttcactctaccccccccccccgaaaataataaaaataataataaaaaagaggaagaataccagcgcaagctttctttgattttagccgctaaaaacagcgtatttgtgcatataagaagaggggattatgtggggattggctgtcagcttggtattgattatcaaaaaaaggcgcttgagtatatggcaaagcgcgtgccaaacatggagctttttgtgttttgcgaagacttaaaattcacgcaaaatcttgatcttggctaccctttcacggacatgaccactagggataaagaagaagaggcgtattgggatatgctgctcatgcaatcttgcaagcatggcattatcgctaatagcacttatagctggtgggcggcttatttgatggaaaatccagaaaaaatcattattggccccaaacactggctttttgggcatgaaaatattctttgtaaggaatgggtgaaaatagaatcccattttgaggtaaaatcccaaaaatataacgcttaa

SEQ ID NO: 10 - DNA, 2850 bp, synthetic construct
  misc_feature 1..2850: note = Fkp
  source 1..2850: mol_type = other DNA
atgcaaaaactactatctttaccgtccaatctggttcagtcttttcatgaactggagagggtgaatcgtaccgattggttttgtacttccgacccggtaggtaagaaacttggttccggtggtggaacatcctggctgcttgaagaatgttataatgaatattcagatggtgctacttttggagagtggcttgaaaaagaaaaaagaattcttcttcatgcgggtgggcaaagccgtcgtttacccggctatgcaccttctggaaagattctcactccggttcctgtgttccggtgggagagagggcaacatctgggacaaaatctgctttctctgcaacttcccctatatgaaaaaatcatgtctttggctccggataaactccatacactgattgcgagtggtgatgtctatattcgttcggagaaacctttgcagagtattcccgaagcggatgtggtttgttatggactgtgggtagatccgtctctggctacccatcatggcgtgtttgcttccgatcgcaaacatcccgaacaactcgactttatgcttcagaagccttcgttggcagaattggaatctttatcgaagacccatttgttcctgatggacatcggtatatggcttttgagtgaccgtgccgtagaaatcttgatgaaacgttctcataaagaaagctctgaagaactaaagtattatgatctttattccgattttggattagctttgggaactcatccccgtattgaagacgaagaggtcaatacgctatccgttgctattctgcctttgccgggaggagagttctatcattacgggaccagtaaagaactgatttcttcaactctttccgtacagaataaggtttacgatcagcgtcgtatcatgcaccgtaaagtaaagcccaatccggctatgtttgtccaaaatgctgtcgtgcggatacctctttgtgccgagaatgctgatttatggatcgagaacagtcatatcggaccaaagtggaagattgcttcacgacatattattaccggggttccggaaaatgactggtcattggctgtgcctgccggagtgtgtgtagatgtggttccgatgggtgataagggctttgttgcccgtccatacggtctggacgatgttttcaaaggagatttgagagattccaaaacaaccctgacgggtattccttttggtgaatggatgtccaaacgcggtttgtcatatacagatttgaaaggacgtacggacgatttacaggcagtttccgtattccctatggttaattctgtagaagagttgggattggtgttgaggtggatgttgtccgaacccgaactggaggaaggaaagaatatctggttacgttccgaacatttttctgcggacgaaatttcggcaggtgccaatctgaagcgtttgtatgcacaacgtgaagagttcagaaaaggaaactggaaagcattggccgttaatcatgaaaaaagtgttttttatcaacttgatttggccgatgcagctgaagattttgtacgtcttggtttggatatgcctgaattattgcctgaggatgctctgcagatgtcacgcatccataaccggatgttgcgtgcgcgtattttgaaattagacgggaaagattatcgtccggaagaacaggctgcttttgatttgcttcgtgacggcttgctggacgggatcagtaatcgtaagagtaccccaaaattggatgtatattccgatcagattgtttggggacgtagccccgtgcgcatcgatatggcagggggatggaccgatactcctccttattcactttattcgggaggaaatgtggtgaatctagccattgagttgaacggacaacctcccttacaggtctatgtgaagccgtgtaaagacttccatatcgtcctgcgttctatcgatatgggtgctatggaaatagtatctacgtttgatgaattgcaagattataagaagatcggttcacctttctctattccgaaagccgctctgtcattggcaggctttgcacctgcgttttctgctgtatcttatgcttcattagaggaacagcttaaagatttcggtgcaggtattgaagtgactttattggctgctattcctgccggttccggtttgggcaccagttccattctggcttctaccgtacttggtgccattaacgatttctgtggtttagcctgggataaaaatgagatttgtcaacgtactcttgttcttgaacaattgctgactaccggaggtggatggcaggatcagtatggaggtgtgttgcagggtatgaagcttcttcagaccgaggccggctttgctcaaagtccattggtgcgttggctacccgatcatttatttacgcatcctgaatacaaagactgtcacttgctttattataccggtataactcgtacggcaaaagggatcttggcagaaatagtcagttccatgttcctcaattcatcgttgcatctcaatttactctcggaaatgaaggcgcatgcattggatatgaatgaagctatacagcgtggaagttttgttgagtttggccgtttggtaggaaaaacctgggaacaaaacaaagcattggatagcggaacaaatcctccggctgtggaggcaattatcgatctgataaaagattataccttgggatataaattgccgggagccggtggtggcgggtacttatatatggtagcgaaagatccgcaagctgctgttcgtattcgtaagatactgacagaaaacgctccgaatccgcgggcacgttttgtcgaaatgacgttatctgataagggattccaagtatcacgatcataa

SEQ ID NO: 11 - DNA, 51 bp, synthetic construct
  misc_feature 1..51: note = fkp forward
  source 1..51: mol_type = other DNA
ctttaataaggagatataccatgcaaaaactactatctttaccgtccaatc

SEQ ID NO: 12 - DNA, 49 bp, synthetic construct
  misc_feature 1..49: note = fkp reverse
  source 1..49: mol_type = other DNA
gcattatgcggccgcaagcttatgatcgtgatacttggaatcccttatc

(19) United States
(12) Patent Application Publication
(10) Pub. No.: US 2024/0309413 A1
(43) Pub. Date: Sep. 19, 2024

(54) GENETICALLY ENGINEERED BACTERIA AND METHODS FOR PREPARING A FUCOSYLATED OLIGOSACCHARIDE USING THE SAME

(71) Applicant: SYNAURA BIOTECHNOLOGY (SHANGHAI) CO., LTD., Shanghai (CN)

(72) Inventors: Zhanbing CHENG, Shanghai (CN); Qi JIAO, Shanghai (CN); Zhenhua TIAN, Shanghai (CN); Shu WANG, Shanghai (CN); Xiaolan XU, Shanghai (CN); Fei YAO, Shanghai (CN); Miao LI, Shanghai (CN); Hong XU, Shanghai (CN); Chenxi HUANG, Shanghai (CN); Yurou LIU, Shanghai (CN)

(73) Assignee: SYNAURA BIOTECHNOLOGY (SHANGHAI) CO., LTD., Shanghai (CN)

(21) Appl. No.: 18/576,663

(22) PCT Filed: Oct. 11, 2022
(86) PCT No.: PCT/CN2022/124634

§ 371 Date: Jan. 4, 2024

(30) Foreign Application Priority Data
Dec. 3, 2021 (CN) 202111468092.2

(51) Int. Cl. C12P 19/00; C12N 9/10; C12N 9/12; C12N 15/52; C12N 15/70

(52) U.S. Cl. CPC C12P 19/00; C12N 9/1051; C12N 9/1205; C12N 9/1241; C12N 15/52; C12N 15/70; C12Y 207/01052; C12Y 207/0703; C12N 2800/101

(57) ABSTRACT

The invention discloses a genetically engineered bacterium and a method for preparing a fucosylated oligosaccharide using the same. The method includes: transferring a fucosyl group of a donor to an oligosaccharide receptor by a fucosyltransferase heterologously expressed in a genetically engineered bacterium; wherein the donor is a nucleotide-activated donor, the fucosyltransferase has α-1,2-fucosyltransferase activity; wherein, the fucosyltransferase is selected from one or more of the enzymes corresponding to NCBI Accession Numbers WP_109047124.1, RTL12957.1, MBP7103497.1, WP_120175093.1, RYE22506.1, WP_140393075.1 and HJB91111.1. The preparation method of the invention has high yield, greatly improved substrate conversion rate and product conversion rate, and has the potential to be applied to industrial production.

### TECHNICAL FIELD

The invention belongs to the field of microbial fermentation, and in particular to a genetically engineered bacterium and a method for preparing a fucosylated oligosaccharide by using the same.

### BACKGROUND OF THE INVENTION

Human milk is composed of a mixture of carbohydrates, proteins, lipids, hormones and trace elements, and can not only provide the nutrients needed for the growth and development of infants, but also provide protective agents such as immunoglobulins. In addition to this, human milk also includes a series of complex oligosaccharides with protective properties—human milk oligosaccharides.

Human milk oligosaccharides (HMOs) are a class of structurally complex non-digestible carbohydrates in human milk, with a content of 22-24 g/L in human colostrum and 5-12 g/L in normal human milk, and are the third most common solid component in human milk after fat and lactose. HMOs balance the development of intestinal flora by stimulating the growth of beneficial intestinal bacteria such as bifidobacteria and lactobacilli in neonates. HMOs may play an important role in regulating the immune system of neonates after birth and are important as a functional component of advanced infant formula food products. In addition, HMOs can inhibit the adhesion of pathogens to glycans on the surface of epithelial cells, thereby limiting the virulence of some pathogens.

There are more than 200 different oligosaccharides in human milk, and the structures of 115 human milk oligosaccharides have been determined. According to the monosaccharide structural units that make up HMOs, HMOs can be classified into three types: neutral fucosyllactose, acidic sialyllactose, and neutral non-fucosylated lactose.

Fucosyltransferase (FucT) is able to catalyze the transfer of fucosyl groups from nucleoside diphosphate fucose (usually GDP-fucose) to receptor molecules (such as oligosaccharides, glycoproteins, glycolipids). Depending on the different addition site of fucosyl group, fucosyltransferases can be classified into α-1,2-fucosyltransferase, α-1,3-fucosyltransferase, α-1,4-fucosyltransferase, α-1,6-fucosyltransferase and O-fucosyltransferase. α-1,2-fucosyltransferases are widely found in vertebrates, invertebrates, plants and bacteria, but the soluble expression level of these fucosyltransferases in most bacteria is very low, which greatly limits biosynthesis of fucosylated oligosaccharides.

At present, the activity of fucosyltransferase in the preparation of fucosylated oligosaccharides is low, which severely limits the production level of fucosylated oligosaccharides and cannot meet the needs of industrial production. Therefore, the invention aims to screen a highly active α-1,2-fucosyltransferase through experimental research, and improve the yield of fucosylated oligosaccharides in commercial production.

### SUMMARY OF THE INVENTION

The technical problems to be solved by the invention are to provide a genetically engineered bacterium and a method for preparing a fucosylated oligosaccharide using the same, in order to overcome the lack of fucosyltransferase with high activity and high yield in the prior art for the industrial production. The genetically engineered bacterium of the invention and the preparation method using the same achieve high yield, greatly improved substrate conversion rate and product conversion rate, and have the potential to be applied to industrial production.

The invention solves the above technical problems through the following technical solutions:

The first aspect of the invention provides a method for preparing a fucosylated oligosaccharide, wherein the method comprises: transferring a fucosyl group of a donor to an oligosaccharide receptor by a fucosyltransferase heterologously expressed in a genetically engineered bacterium; wherein the donor is a nucleotide-activated donor, and the fucosyltransferase has α-1,2-fucosyltransferase activity;
wherein, the fucosyltransferase is selected from one or more of enzymes corresponding to NCBI Accession Numbers WP_109047124.1, RTL12957.1, MBP7103497.1, WP_120175093.1, RYE22506.1, WP_140393075.1 and HJB91111.1.

In some embodiments of the invention, the fucosyltransferases are the enzymes corresponding to NCBI Accession Number RTL12957.1 and WP_120175093.1.

In some embodiments of the invention, the oligosaccharide receptor is selected from lactose, 3-fucosyllactose, lacto-N-tetraose, lacto-N-neotetraose, lacto-N-fucosylpentose II, lacto-N-hexose and sialyllacto-N-tetraose b.

In some embodiments of the invention, the fucosylated oligosaccharide is selected from 2′-fucosyllactose, 2′,3-difucosyllactose, lacto-N-fucosylpentose I, lacto-N-neofucosylpentose I, lacto-N-difucosylhexose I, lacto-N-fucosylheptose I and fucosyllacto-N-sialylpentose b.

In some specific embodiments of the invention, the donor is guanosine diphospho-fucose.

In some embodiments of the invention, the genetically engineered bacterium is an engineered Escherichia coli (E. coli) or yeast.

In some preferred embodiments of the invention, the genetically engineered bacterium is an engineered E. coli BL21 (DE3) strain.

In some embodiments of the invention, the genetically engineered bacterium also expresses a bifunctional enzyme with both L-fucokinase/fucose-1-phosphate guanosyltransferase functions; preferably, the bifunctional enzyme is an enzyme corresponding to NCBI Accession Number WP_010993080.1.

Additionally or alternatively, in the genetically engineered bacterium, an bypass metabolic pathway of the oligosaccharide receptor is inhibited; preferably, the bypass metabolic pathway of the oligosaccharide receptor is inhibited by knocking out or mutating a gene; more preferably, when the oligosaccharide receptor is lactose, a gene encoding β-galactosidase in the genetically engineered bacterium, such as lacZ gene, is knocked out and inactivated, and the metabolic pathway of lactose degradation to galactose is inhibited.

In the invention, the bypass metabolic pathway of the oligosaccharide receptor refers to a metabolic pathway other than as the fucosyl receptor.

Additionally or alternatively, in the genetically engineered bacterium, an bypass metabolic pathway of the precursor of the donor is inhibited; preferably, the bypass metabolic pathway of the precursor is inhibited by knocking out or mutating a gene; more preferably, when the donor is guanosine diphospho-fucose, the precursor is L-fucose, and the genes encoding L-fucose isomerase and/or L-fucokinase in the genetically engineered bacterium, such as FucI and/or FucK, are knocked out and inactivated, and the bypass metabolic pathway of L-fucose is inhibited.

In the invention, the bypass metabolic pathway of the precursor of the donor refers to a metabolic pathway other than conversion into the donor.

And/or, in the genetically engineered bacterium, an bypass metabolic pathway of the donor is inhibited; preferably, the bypass metabolic pathway of the donor is inhibited by knocking out or mutating a gene; more preferably, when the donor is guanosine diphospho-fucose, a gene encoding UDP-glucose lipid carrier transferase in the genetically engineered bacterium, such as wacJ, is knocked out and inactivated, and the competitive utilization pathway of degradation of guanosine diphospho-fucose to colanic acid is blocked.

In the invention, the bypass metabolic pathway of the donor refers to a metabolic pathway other than providing the fucosyl group.

In some embodiments of the invention, the method further comprises the fermentation culture of the genetically engineered bacterium in a fermentation medium.

Preferably, the fermentation medium comprises: 20-25 g/L of glycerol, 10-12 g/L of peptone, 5-6 g/L of yeast powder, 10-12 g/L of NaCl. 0.1-0.2 mM of IPTG, 5-6 g/L of precursor molecules for synthesizing the donor such as L-fucose, and 10-15 g/L of oligosaccharide receptor such as lactose are added when the OD₆₀₀ of the fermentation medium is 0.6-0.8; and/or, the conditions of the fermentation culture are: 25-27° C., 220 r/min.

The second aspect of the invention provides a genetically engineered bacterium expressing a fucosyltransferase, wherein the fucosyltransferase has α-1,2-fucosyltransferase activity; the fucosyltransferase transfers a fucosyl group of a donor to an oligosaccharide receptor, and the donor is a nucleotide-activated donor; Wherein, the fucosyltransferase is one or more of enzymes corresponding to NCBI Accession Numbers WP_109047124.1, RTL12957.1, MBP7103497.1, WP_120175093.1, RYE22506.1, WP_140393075.1 and HJB91111.1.

The oligosaccharide receptor, the fucosylated oligosaccharide and the donor are preferably as defined in the first aspect.

In some embodiments of the invention, the genetically engineered bacterium is an engineered E. coli or yeast; preferably, the genetically engineered bacterium is an engineered E. coli BL21 (DE3) strain.

In some embodiments of the invention, the genetically engineered bacterium expresses a bifunctional enzyme with both L-fucokinase/fucose-1-phosphate guanosyltransferase; preferably, the bifunctional enzyme is an enzyme corresponding to NCBI Accession Number WP_010993080.1.

And/or, in the genetically engineered bacterium, a bypass metabolic pathway of the oligosaccharide receptor is inhibited; preferably, the bypass metabolic pathway of the oligosaccharide receptor is inhibited by knocking out or mutating a gene; more preferably, when the oligosaccharide receptor is lactose, the gene encoding β-galactosidase in the genetically engineered bacterium, such as lacZ gene, is knocked out and inactivated, and the metabolic pathway of lactose degradation to galactose is inhibited.

And/or, in the genetically engineered bacterium, a bypass metabolic pathway of the precursor of the donor is inhibited; preferably, the bypass metabolic pathway of the precursor is inhibited by knocking out or mutating a gene; more preferably, when the donor is guanosine diphospho-fucose, the precursor is L-fucose, and the genes encoding L-fucose isomerase and/or L-fuculokinase in the genetically engineered bacterium, such as FucI and/or FucK, are knocked out and inactivated, and the bypass metabolic pathway of L-fucose is inhibited.

And/or, in the genetically engineered bacterium, a bypass metabolic pathway of the donor is inhibited; preferably, the bypass metabolic pathway of the donor is inhibited by knocking out or mutating a gene; more preferably, when the donor is guanosine diphospho-fucose, the gene encoding UDP-glucose lipid carrier transferase in the genetically engineered bacterium, such as wacJ, is knocked out and inactivated, and the competitive utilization pathway of guanosine diphospho-fucose degradation to colanic acid is blocked.

The third aspect of the invention provides a method for preparing a fucosylated oligosaccharide, the method comprising:
providing a fucosyltransferase having α-1,2-fucosyltransferase activity in the reaction system, wherein the fucosyltransferase transfers a fucosyl group of a nucleotide-activated donor to an oligosaccharide receptor;
wherein, the fucosyltransferase is one or more of enzymes corresponding to NCBI Accession Numbers WP_109047124.1, RTL12957.1, MBP7103497.1, WP_120175093.1, RYE22506.1, WP_140393075.1 and HJB91111.1.

In some embodiments of the invention, a bifunctional enzyme having both L-fucokinase and fucose-1-phosphate guanyltransferase activities, for example, the enzyme corresponding to NCBI Accession Number WP_010993080.1 is also provided in the reaction system.

The fourth aspect of the invention provides a combination of enzymes comprising two or more selected from fucosyltransferases corresponding to NCBI Accession Numbers WP_109047124.1, RTL12957.1, MBP7103497.1, WP_120175093.1, RYE22506.1, WP_140393075.1 and HJB91111.1.

Alternatively, the combination of enzymes comprises one or more selected from fucosyltransferases corresponding to NCBI Accession Numbers WP_109047124.1, RTL12957.1, MBP7103497.1, WP_120175093.1, RYE22506.1, WP_140393075.1 and HJB91111.1, and further comprises a bifunctional enzyme with both L-fucokinase/fucose-1-phosphate guanyltransferase, preferably the enzyme corresponding to NCBI Accession Number WP_010993080.1

In the invention, the nucleotide sequence encoding the enzyme corresponding to NCBI Accession Number WP_109047124.1 is preferably as set forth in SEQ ID NO: 1; the nucleotide sequence encoding the enzyme corresponding to NCBI Accession Number RTL12957.1 is preferably as set forth in SEQ ID NO: 2; the nucleotide sequence encoding the enzyme corresponding to NCBI Accession Number MBP7103497.1 is preferably as set forth in SEQ ID NO: 3; the nucleotide sequence encoding the enzyme corresponding to NCBI Accession Number RYE22506.1 is preferably as set forth in SEQ ID NO: 4; the nucleotide sequence encoding the enzyme corresponding to NCBI Accession Number WP_120175093.1 is preferably as set forth in SEQ ID NO: 5; the nucleotide sequence encoding the enzyme corresponding to NCBI Accession Number WP_140393075.1 is preferably as set forth in SEQ ID NO: 6; the nucleotide sequence encoding the enzyme corresponding to NCBI Accession Number HJB91111.1 is preferably as set forth in SEQ ID NO: 7; and the nucleotide sequence encoding the enzyme corresponding to NCBI Accession Number WP_010993080.1 is preferably as set forth in SEQ ID NO: 10.

The fifth aspect of the invention provides the use of a fucosyltransferase or th combination of enzymes as described in the fourth aspect in the preparation of a fucosylated oligosaccharide, wherein the fucosyltransferase is an enzyme corresponding to NCBI Accession Number WP_109047124.1, RTL12957.1, MBP7103497.1, WP_120175093.1, RYE22506.1, WP_140393075.1, HJB91111.1, or MBE2189475.1.

In the invention, the oligosaccharide receptor and the fucosylated oligosaccharide are preferably as shown in Table 1 below:

TABLE 1
Oligosaccharide receptors and fucosylated oligosaccharides

| | Name | Abbreviation | Structure |
|---|---|---|---|
| Oligosaccharide | lactose | Lac | Gal(β1,4)Glc |
| receptors | 3-fucosyllactose | 3-FL | Gal(β1,4)[Fuc(α1,3)]Glc |
| | lacto-N-tetraose | LNT | Gal(β1,3)GlcNAc(β1,3)Gal(β1,4)Glc |
| | lacto-N-neotetraose | LNnT | Gal(β1,4)GlcNAc(β1,3)Gal(β1,4)Glc |
| | lacto-N-fucosylpentose | LNFP II | Gal(β1,3)[Fuc(α1,4)]GlcNAc(β1,3)Gal(β1,4)Glc |
| | II | | |
| | sialyllacto-N-tetraose | LSTb | Gal(β1,3)[Neu5Ac(α2,6)]GlcNAc(β1,3)Gal(β1,4)Glc |
| | b | | |
| | lacto-N-hexose | LNH | Gal(β1,4)GlcNAc(β1,6)[Gal(β1,3)GlcNAc(β1,3)]Gal(β1,4)Glc |
| Fucosylated | 2′-fucosyllactose | 2′-FL | Fuc(α1,2)Gal(β1,4)Glc |
| oligosaccharides | 2′,3-difucosyllactose | 2′,3-FL | Fuc(α1,2)Gal(β1,4)[Fuc(α1,3)]Glc |
| | lacto-N-fucosylpentose | LNFP I | Fuc(α1,2)Gal(β1,3)GlcNAc(β1,3)Gal(β1,4)Glc |
| | I | | |
| | lacto-N-neofucosylpentose | LNnFP I | Fuc(α1,2)Gal(β1,4)GlcNAc(β1,3)Gal(β1,4)Glc |
| | I | | |
| | lacto-N-difucosylhexose | LNDH I | Fuc(α1,2)Gal(β1,3)[Fuc(α1,4)]GlcNAc(β1,3)Gal |
| | I | | (β1,4)Glc |
| | fucosyllacto-N-sialylpentose | F-LST-b | Fuc(α1,2)Gal(β1,3)[Neu5Ac(α2,6)]GlcNAc(β1,3)Gal(β1,4)Glc |
| | b | | |
| | lacto-N-fucosylheptose | F-LNH I | Fuc(α1,2)Gal(β1,3)GlcNAc(β1,3)[Gal(β1,4)Glc |
| | I | | NAc(β1,6)]Gal(β1,4)Glc |

On the basis of conforming to common knowledge in the art, the above preferred conditions can be combined arbitrarily to obtain preferred examples of the invention.

The reagents and raw materials used in the invention are all commercially available.

The positive progressive effects of the invention lie in:
the genetically engineered bacterium of the invention and the method for preparation of fucosylated oligosaccharides using the same have high yield, greatly improved substrate conversion rate and product conversion rate, and have the potential to be applied to industrial production.

### EXAMPLES

The invention is further described below by Examples, but the invention is not limited to the scope of the Examples. The experimental methods that do not indicate specific conditions in the following Examples are selected according to conventional methods and conditions, or according to the product instruction.

The experimental methods in the invention are conventional methods unless otherwise indicated, and the gene cloning operation may be specifically found in “Molecular Cloning: A Laboratory Manual” edited by J. Sambrook et al.

pET28a/pCDFduet-1 was purchased from Novagen Company; competent E. coli BL21 (DE3) cells were purchased from Thermo Fisher Company, and competent E. coli DH5a cells were purchased from Beijing Dingguo Changsheng Biotechnology Co. Ltd., endonuclease was commercially available, lactose was purchased from Sinopharm Reagent, L-fucose was purchased from Carbosynth, and seamless cloning kit ClonExpress II One Step Cloning Kit was purchased from Novozymes.

A high-performance liquid chromatography (HPLC) system (SHIMADZULC-20ADXR) was used to quantitatively detect the synthesis of 2′-FL in the fermentation broth of recombinant E. coli in the Examples, and the concentrations of 2′-FL and the substrate lactose in the fermentation broth were determined by HP-Amide column (Sepax, 4.6×250 mm 5 μm). The HPLC detector was a differential detector, the detection temperature of the chromatographic column was set to 35° C., the mobile phase was eluted by acetonitrile:water=68:32, and the detection flow rate was 1.4 mL/min.

### Example 1 Obtaining FucT Gene and Preparation of FucT Crude Enzyme Solution

The sequences of α-1,2-fucosyltransferase gene FucT published on NCBI were totally synthesized and inserted into the vector pCDFduet-1 at the restriction sites NcoI and HindIII to construct the recombinant plasmid pCDFduet-1-FucT. The sequences for total synthesis are shown in Table 2, and the gene synthesis company is Suzhou Genewiz Biotechnology Co., Ltd. (Floor C3, Bio-Nano Technology Park, No. 218, Xinghu Street, Suzhou Industrial Park).

TABLE 2
Synthesized gene sequences and related information

| Enzyme | | | | SEQ |
|---|---|---|---|---|
| Name | No. | GenBank No | Species Origin | ID NO: |
| AzoFucT | GT062 | WP_109047124.1 | Azospirillum sp. TSA6c | 1 |
| NeiFucT | GT065 | RTL12957.1 | Neisseriaceae bacterium | 2 |
| BacFucT | GT072 | MBP7103497.1 | Bacteroidales bacterium | 3 |
| SphFucT | GT083 | RYE22506.1 | Sphingobacteriaceae bacterium | 4 |
| PreFucT | GT093 | WP_120175093.1 | Prevotella melaninogenica | 5 |
| LacFucT | GT104 | WP_140393075.1 | Lachnoclostridium sp. An138 | 6 |
| CeiFucT | GT107 | HJB91111.1 | Candidatus Eisenbergiella | 7 |
| | | | merdigallinarum | |
| CkaFucT | GT059 | MBE2189475.1 | Candidatus Kapabacteria | 8 |
| HpFucT | HpFucT | AAC99764.1 | Helicobacter pylori UA802 | 9 |
| ProbF | fkp | WP_010993080.1 | Bacteroides fragilis | 10 |

The above gene vectors were transformed into competent host E. coli BL21(DE3) cells respectively; the recombinant cells comprising pCDFduet-1-FucT vectors were inoculated into LB liquid medium containing 30 μg/mL spectinomycin, and cultured in a shaker at 200 rpm at 37° C. The culture was added IPTG to a final concentration of 0.05 mM when OD₆₀₀ reaches 0.8-1.0, and cooled to 30° C. for overnight induction. At the end of the fermentation, the culture was centrifuged at 5000 rpm for 20 min to remove the fermentation broth and retain the bacterial cells.

5 g of bacterial cells were resuspended by adding 50 mL of phosphate buffer (pH 7.0, 25 mM), homogenized and broken at 4° C. and 800 mbar for 3 min, and then centrifuged at 5000 rpm and 15° C. for 30 min. The supernatant was retained to prepare the crude enzyme liquid, which was placed at 4° C. for purification.

The composition of LB liquid medium: 10 g/L of peptone, 5 g/L of yeast powder, and 10 g/L of NaCl were dissolved in deionized water and then metered volume, sterilized at 121° C. for 20 min, and put aside.

### Example 2 Purification and Enzyme Activity Analysis of FucT Enzymes

### Purification of Enzymes

The purification steps are as follows: the Ni column stored at 4° C. was taken, the closed column head was opened, and the original column liquid was drained. The Ni column was rinsed with 50 mL of deionized water. The Ni column was rinsed with 10 mL of 1× Binding Buffer. The crude enzyme solution prepared in Example 1 was loaded onto the column twice. The Ni column was rinsed with 10 mL of Binding Buffer (containing 20 mM imidazole). The Ni column was rinsed with 10 mL Wash Buffer (containing 40 mM imidazole). The impurity proteins were eluted using 5 mL of Elution Buffer (containing 80 mM imidazole), and then pure protein was eluted using 5 mL of Elution Buffer (containing 250 mM imidazole). 10 kDa Millipore ultrafiltration concentrator tubes were used for concentration and removing salts. Pure FucT may be obtained after protein purification by SDS PAGE.

### Enzyme Activity Assay of FucT

The reaction conditions are as follows: the reaction with a total volume of 50 μL comprising a final concentration of 25 mM phosphate buffer (pH 5.6), 5 mM GDP-fucose, 10 mM lactose, 1 mg/mL FucT pure enzyme, was reacted at 37° C. for 20 min. The reaction was terminated in a boiling water bath for 10 min, centrifuged at 12,000 rpm for 5 min, and the supernatant was collected for HPLC analysis, the final concentration of the product was determined using the external standard method, and the enzyme activity and specific enzyme activity were calculated. The enzyme activity of 1 U was defined as the amount of enzyme required to produce 1 μmol of 2′-FL per minute in the above reaction system. The experimental data of specific enzyme activity are shown in Table 3 below.

TABLE 3
Specific enzyme activity data

| | Specific Enzyme |
|---|---|
| Enzyme No. | Activity U/mg |
| GT062 | 615 |
| GT065 | 532 |
| GT072 | 413 |
| GT083 | 397 |
| GT093 | 459 |
| GT104 | 411 |
| GT107 | 566 |
| GT059 | 113 |
| HpFucT | 85 |

### Example 3 Preparation of FucT and fkp Genes Co-Expression Vector

The sequence of bifunctional gene L-fucokinase/fucose-1-phosphate guanosyltransferase gene fkp published on NCBI (see Table 2) was totally synthesized and ligated into the vector pET28a at the restriction sites NcoI and HindIII. The gene synthesis company is Suzhou Genewiz Biotechnology Co., Ltd. (Floor C3, Bio-Nano Technology Park, No. 218, Xinghu Street, Suzhou Industrial Park). The fkp gene was obtained.

The fkp gene was cloned into the second reading frame position of each pCDFduet-1-FucT plasmid prepared in Example 1 at the restriction sites NdeI and XhoI, and a series of co-expression vectors as shown in the Table 4 were constructed with a seamless cloning kit. The list of primers is shown in Table 5. The above co-expression plasmid vectors containing fkp and FucT were transformed into the competent host E. coli DH5a cells to obtain recombinant genetically engineered strains. For the specific operation method of vector construction, please see the kit instruction manual of ClonExpress II One Step Cloning Kit.

TABLE 4
List of co-expression vectors

| Enzyme No. | Vector Name | GenBank No. |
|---|---|---|
| GT062 | pCDF -AzoFucT-fkp | WP_109047124.1 |
| GT065 | pCDF -NeiFucT-fkp | RTL12957.1 |
| GT072 | pCDF -BacFucT-fkp | MBP7103497.1 |
| GT083 | pCDF -SphFucT-fkp | RYE22506.1 |
| GT093 | pCDF -PreFucT-fkp | WP_120175093.1 |
| GT104 | pCDF -LacFucT-fkp | WP_140393075.1 |
| GT107 | pCDF -CeiFucT-fkp | HJB91111.1 |
| GT059 | pCDF -CkaFucT-fkp | MBE2189475.1 |
| HpFucT | pCDF -HpFucT-fkp | AAC99764.1 |
| (Control) | | |

TABLE 5
List of fkp primer sequences

| fkp primer | Primer Sequence | SEQ ID NO: |
|---|---|---|
| fkp forward | ctttaataaggagatataccatgcaaaaactactatctttaccgtccaatc | 11 |
| fkp reverse | gcattatgcggccgcaagcttatgatcgtgatacttggaatcccttatc | 12 |

### Example 4 Engineering of E. coli BL21(DE3) Strain

In this Example, E. coli BL21 (DE3) was used as the parental host to construct a strain for whole-cell biosynthesis of 2′-fucosyllactose. The genome engineering includes gene break and deletion.

The biosynthesis of 2′-fucosyllactose was performed using lactose as the receptor substrate, L-fucose as the precursor of the glycosyl donor, and GDP-L-fucose as the glycosyl donor. Therefore, the lacZ gene encoding β-galactosidase in the host cell was first inactivated in this Example (Qi Li, Bingbing Sun, Jun Chen, Yiwen Zhang, Yu Jiang, Sheng Yang, A modified pCas/pTargetF system for CRISPR-Cas9-assisted genome editing in Escherichia coli, Acta Biochimica et Biophysica Sinica, Volume 53, Issue 5, May 2021, Pages 620-627), to prevent degradation of the substrate lactose; the FucI gene and fucK gene encoding L-fucose isomerase and/or L-fuculokinase were secondly deleted using the same method, to prevent the degradation of L-fucose; the wacJ gene encoding UDP-glucose lipid carrier transferase was deleted in the third step to block the competitive utilization pathway of guanosine diphospho-fucose degradation to colanic acid (Dumon, C., Priem, B., Martin, S. L. et al. In vivo fucosylation of lacto-N-neotetraose and lacto-N-neohexaose by heterologous expression of Helicobacter pylori α-1,3 fucosyltransferase in engineered Escherichia coli. Glycoconj J 18, 465-474 (2001)). Finally, a strain of BL21(DE3)lacZ(ΔM15)ΔfucK-fucIΔwacJ was obtained.

### Example 5 Preparation of 2′-Fucosyllactose by Fermentation

A series of co-expression vector plasmids described in Table 4 in Example 3 were respectively transformed into the strain of BL21(DE3)lacZ(ΔM15)ΔfucK-fucIΔwacJ described in Example 4, and recovered at 37° C. for 1 h and spread on a LB plates with spectinomycin-resistant at final concentration of 25 μg/mL, cultured at 37° C. for 10-12 h to obtain the fermentation recombinant bacteria containing fkp and FucT genes.

Single colonies were picked up and cultured in LB medium with a final concentration of 25 μg/mL spectinomycin for 8-10 h, and used as the seed liquid for fermentation in shaking flask.

The seed liquid was then inoculated into a 250 mL conical flask containing 100 mL of fermentation medium at an inoculum amount of 1%, and spectinomycin at a final concentration of 25 μg/mL was added at the same time. The formula of the fermentation medium was: 20 g/L of glycerol, 10 g/L of peptone, 5 g/L of yeast powder, 10 g/L of NaCl; the volume was adjusted with deionized water. Subsequently, when the flask was cultured at 25° C. and 220 r/min until OD₆₀₀=0.6-0.8, IPTG at a final concentration of 0.1 mM, L-fucose at a final concentration of 5 g/L, and lactose at a final concentration of 10 g/L were added, and fermentation was preformed continuously for 72 h.

At the end of fermentation, the yield of extracellular 2′-fucosyllactose (2′-FL) and the remaining amounts of lactose and fucose were determined by using high performance liquid chromatography (HPLC).

First, 2 mL of the fermentation broth was centrifuged at 12,000 rpm for 10 min, and the supernatant was collected, passed through a 0.22 μm filter membrane, and the concentrations of extracellular 2′-fucosyllactose, lactose, and L-fucose were detected by HPLC. The results are shown in Table 6 below.

TABLE 6
Results of fermentation experiments

| | | Dry | | | | | |
|---|---|---|---|---|---|---|---|
| | | weight | | | | Conversion | Conversion |
| | | of cells | | | | rate of | rate of |
| | | at the | Lactose | Fucose | 2′-FL | lactose | fucose |
| | | end of | consump- | consump- | maximum | (mole | (mole |
| Enzyme | | fermentation | tion | tion | yield | 2′-FL/mol | 2′-FL/mol |
| No. | Vector name | (g/L) | (g/L) | (g/L) | (g/L) | lactose) | fucose) |
| GT062 | pCDF-AzoFucT-fkp | 4.36 | 3.78 | 1.22 | 1.78 | 0.33 | 0.49 |
| GT065 | pCDF-NeiFucT-fkp | 4.45 | 5.47 | 1.67 | 2.89 | 0.37 | 0.58 |
| GT072 | pCDF-BacFucT-fkp | 4.28 | 3.76 | 1.32 | 2.04 | 0.38 | 0.52 |
| GT083 | pCDF-SphFucT-fkp | 4.79 | 5.67 | 1.44 | 2.35 | 0.29 | 0.55 |
| GT093 | pCDF-PreFucT-fkp | 4.06 | 8.38 | 2.12 | 3.35 | 0.28 | 0.53 |
| GT104 | pCDF-LacFucT-fkp | 4.12 | 4.44 | 1.46 | 2.22 | 0.35 | 0.51 |
| GT107 | pCDF-CeiFucT-fkp | 4.57 | 4.23 | 1.36 | 1.75 | 0.29 | 0.43 |
| GT059 | pCDF-CkaFucT-fkp | 4.34 | 0.9 | 0.43 | 0.36 | 0.28 | 0.28 |
| HpFucT | pCDF-HpFucT-fkp | 4.22 | 1.5 | 0.44 | 0.75 | 0.35 | 0.57 |
| (control) | | | | | | | |

As shown in the above table, except for GT059, the yield of 2′-FL obtained by fermentation of other strains in the recombinant strains was much higher than that of the control group.

1. A method for preparing a fucosylated oligosaccharide, wherein the method comprises: transferring a fucosyl group of a donor to an oligosaccharide receptor by a fucosyltransferase heterologously expressed in a genetically engineered bacterium; wherein the donor is a nucleotide-activated donor, and the fucosyltransferase has α-1,2-fucosyltransferase activity;
wherein the fucosyltransferase is an enzyme corresponding to NCBI Accession Number RTL12957.1 or WP 120175093.1;
wherein the genetically engineered bacterium further expresses a bifunctional enzyme with both L-fucokinase and fucose-1-phosphate guanyltransferase activities and the bifunctional enzyme is an enzyme corresponding to NCBI Accession Number WP 010993080.1.

2. The method of claim 1, wherein the oligosaccharide receptor is selected from the group consisting of lactose, 3-fucosyllactose, lacto-N-tetraose, lacto-N-neotetraose, lacto-N-fucosylpentose II, lacto-N-hexose and sialyllacto-N-tetraose b;
and/or, the fucosylated oligosaccharide is selected from the group consisting of 2′-fucosyllactose, 2′,3-difucosyllactose, lacto-N-fucosylpentose I, lacto-N-neofucosylpentose I, lacto-N-difucosylhexose I, lacto-N-fucosylheptose I and fucosyllacto-N-sialylpentose b;
and/or, the donor is guanosine diphospho-fucose;
and/or, the genetically engineered bacterium is an engineered Escherichia coli (E. coli) or yeast; preferably, the genetically engineered bacterium is an engineered E. coli BL21 (DE3) strain.

3. The method of claim 1, wherein
in the genetically engineered bacterium, a bypass metabolic pathway of the oligosaccharide receptor is inhibited; preferably, the bypass metabolic pathway of the oligosaccharide receptor is inhibited by knocking out or mutating a gene; more preferably, when the oligosaccharide receptor is lactose, a gene encoding β-galactosidase in the genetically engineered bacterium, such as lacZ gene, is knocked out and inactivated, and a metabolic pathway of lactose degradation to galactose is inhibited;
and/or, in the genetically engineered bacterium, a bypass metabolic pathway of a precursor of the donor is inhibited; preferably, the bypass metabolic pathway of the precursor is inhibited by knocking out or mutating a gene; more preferably, when the donor is guanosine diphospho-fucose, the precursor is L-fucose, and genes encoding L-fucose isomerase and/or L-fuculokinase in the genetically engineered bacterium, such as FucI and/or FucK, are knocked out and inactivated, and the bypass metabolic pathway of L-fucose is inhibited;
and/or, in the genetically engineered bacterium, a bypass metabolic pathway of the donor is inhibited; preferably, the bypass metabolic pathway of the donor is inhibited by knocking out or mutating a gene; more preferably, when the donor is guanosine diphospho-fucose, a gene encoding UDP-glucose lipid carrier transferase in the genetically engineered bacterium, such as wacJ, is knocked out and inactivated, and the competitive utilization pathway of guanosine diphospho-fucose degradation to colanic acid is blocked.

4. The method of claim 1, wherein the method further comprises the fermentation culture of the genetically engineered bacterium in a fermentation medium;
preferably, the fermentation medium comprises: 20-25 g/L of glycerol, 10-12 g/L of peptone, 5-6 g/L of yeast powder, 10-12 g/L of NaCl, as well as 0.1-0.2 mM of IPTG, 5-6 g/L of a precursor molecule for synthesizing the donor such as L-fucose, and 10-15 g/L of oligosaccharide such as lactose which are added when the OD₆₀₀ of the fermentation medium is 0.6-0.8; and/or, the condition of the fermentation culture is: 25-27° C. and 220 r/min.

5. A genetically engineered bacterium heterologously expressing a fucosyltransferase, wherein the fucosyltransferase has α-1,2-fucosyltransferase activity; the fucosyltransferase transfers a fucosyl group of a donor to an oligosaccharide receptor, and the donor is a nucleotide-activated donor;
wherein, the fucosyltransferase is an enzyme corresponding to NCBI Accession Number RTL12957.1 or WP 120175093.1;
wherein the genetically engineered bacterium further expresses a bifunctional enzyme with both L-fucokinase and fucose-1-phosphate guanyltransferase activities and the bifunctional enzyme is an enzyme corresponding to NCBI Accession Number WP 010993080.

6. The genetically engineered bacterium of claim 5, wherein the oligosaccharide receptor is selected from the group consisting of lactose, 3-fucosyllactose, lacto-N-tetraose, lacto-N-neotetraose, lacto-N-fucosylpentose II, lacto-N-hexose and sialyllacto-N-tetraose b;
and/or, the fucosylated oligosaccharide is selected from the group consisting of 2′-fucosyllactose, 2′,3-difucosyllactose, lacto-N-fucosylpentose I, lacto-N-neofucosylpentose I, lacto-N-difucosylhexose I, lacto-N-fucosylheptose I and fucosyllacto-N-sialylpentose b;
and/or, the donor is guanosine diphospho-fucose;
and/or, the genetically engineered bacterium is an engineered E. coli or yeast; preferably, the genetically engineered bacterium is an engineered E. coli BL21 (DE3) strain.

7. The genetically engineered bacterium of claim 5, wherein the nucleotide sequence encoding the fucosyltransferase is set forth in any one of SEQ ID NOs: 2 and 5 and/or the nucleotide sequence encoding the bifunctional enzyme is set forth in SEQ ID NO: 10;
and/or, in the genetically engineered bacterium, a bypass metabolic pathway of the oligosaccharide receptor is inhibited; preferably, the bypass metabolic pathway of the oligosaccharide receptor is inhibited by knocking out or mutating a gene; more preferably, when the oligosaccharide receptor is lactose, a gene encoding β-galactosidase in the genetically engineered bacterium, such as lacZ gene, is knocked out and inactivated, and the metabolic pathway of lactose degradation to galactose is inhibited;
and/or, in the genetically engineered bacterium, a bypass metabolic pathway of a precursor of the donor is inhibited; preferably, the bypass metabolic pathway of the precursor is inhibited by knocking out or mutating a gene; more preferably, when the donor is guanosine diphospho-fucose, the precursor is L-fucose, and genes encoding L-fucose isomerase and/or L-fuculokinase in the genetically engineered bacterium, such as FucI and/or FucK, are knocked out and inactivated, and the bypass metabolic pathway of L-fucose is inhibited;
and/or, in the genetically engineered bacterium, a bypass metabolic pathway of the donor is inhibited; preferably, the bypass metabolic pathway of the donor is inhibited by knocking out or mutating a gene; more preferably, when the donor is guanosine diphospho-fucose, a gene encoding UDP-glucose lipid carrier transferase in the genetically engineered bacterium, such as wacJ, is knocked out and inactivated, and the competitive utilization pathway of guanosine diphospho-fucose degradation to colanic acid is blocked.

8. A method for preparing a fucosylated oligosaccharide, wherein the method comprises:
providing a fucosyltransferase having α-1,2-fucosyltransferase activity in a reaction system, the fucosyltransferase transfers a fucosyl group of a nucleotide-activated donor to an oligosaccharide receptor;
wherein, the fucosyltransferase is selected from one or more of enzymes corresponding to NCBI Accession Number RTL12957.1 or WP 120175093.1;
further providing a bifunctional enzyme having both L-fucokinase and fucose-1-phosphate guanyltransferase activities, in the reaction system, wherein the bifunctional enzyme corresponds to NCBI Accession Number WP 010993080.1.

9. (canceled)

10. (canceled)

11. The method of claim 2, wherein the genetically engineered bacterium is an engineered E. coli BL21 (DE3) strain, in which lacZ gene, FucI FucK, and wacJ are knocked out.

12. The method of claim 2, wherein the method further comprises the fermentation culture of the genetically engineered bacterium in a fermentation medium;
preferably, the fermentation medium comprises: 20-25 g/L of glycerol, 10-12 g/L of peptone, 5-6 g/L of yeast powder, 10-12 g/L of NaCl, as well as 0.1-0.2 mM of IPTG, 5-6 g/L of a precursor molecule for synthesizing the donor such as L-fucose, and 10-15 g/L of oligosaccharide such as lactose which are added when the OD₆₀₀ of the fermentation medium is 0.6-0.8; and/or, the condition of the fermentation culture is: 25-27° C. and 220 r/min.

13. The method of claim 3, wherein the method further comprises the fermentation culture of the genetically engineered bacterium in a fermentation medium;
preferably, the fermentation medium comprises: 20-25 g/L of glycerol, 10-12 g/L of peptone, 5-6 g/L of yeast powder, 10-12 g/L of NaCl, as well as 0.1-0.2 mM of IPTG, 5-6 g/L of a precursor molecule for synthesizing the donor such as L-fucose, and 10-15 g/L of oligosaccharide such as lactose which are added when the OD₆₀₀ of the fermentation medium is 0.6-0.8; and/or, the condition of the fermentation culture is: 25-27° C. and 220 r/min.

14. The genetically engineered bacterium of claim 5, wherein the genetically engineered bacterium is an engineered E. coli BL21 (DE3) strain, in which lacZ gene, FucI FucK, and wacJ are knocked out.
